(19) United States
(12) Patent Application Publication
(10) Pub. No.: US 2008/0147151 A1
(43) Pub. Date: Jun. 19, 2008

(54) Thermal cooling apparatus and related methods

(76) Inventor: Robert Rivera, North Miami Beach, FL (US)

Correspondence Address: BERENATO, WHITE & STAVISH, LLC, 6550 ROCK SPRING DRIVE, SUITE 240, BETHESDA, MD, 20817, US

(21) Appl. No.: 11/639,271

(22) Filed: Dec. 15, 2006

(51) Int. Cl. A61F 7/10; F25D 3/00

(52) U.S. Cl. 607/112; 607/114; 62/530

(57) ABSTRACT

A thermal cooling apparatus is provided that includes a thermal pack and a flexible band conformable into an annulus. The flexible band includes outer and inner layers made of thermally insulating material. The inner and outer layers are connected to one another to establish a pocket including a mouth sized to permit insertion and removal of the thermal pack through the mouth for accommodation in the pocket. The inner layer preferably possesses a plurality of vent ports for permitting thermal transfer from the thermal pack through the inner layer. Also provided is a method of treating an anatomical area of an individual with the thermal cooling apparatus.

### FIELD OF THE INVENTION

The present invention relates to a thermal cooling apparatus and related methods of making and using the same.

### BACKGROUND OF THE INVENTION

The use of cold packs for therapeutic applications is well known. Cold packs are particularly prevalent for reducing high body temperatures, and cooling off on a hot day, or otherwise generally comforting the user, including providing relief from headaches. It is also known to use cold packs for treatment of sports injuries. For athletic purposes, cold packs typically are applied to an injured anatomical area to reduce swelling and inflammation or to prevent their onset, such as might be caused by a twisted joint, cramp, or hyper-extended muscle, or inflicted by a traumatic impact.

A reusable cold pack generally comprises a freezable liquid or gel material disposed within a sealed plastic container or flexible bag. The cold pack is stored in a cool environment, such as a refrigerator or freezer, often below the freezing point of the gel or liquid. When needed for cooling or treatment purposes, the cold pack is removed from the cool environment and applied to the injured anatomical area of the user's body. Heat transfer between the cold pack and the user's body provides the desired therapeutic results, after which the cold pack is returned to the cool environment or is disposed of if not reusable.

One of the main problems encountered with the use of cold packs is the uncomfortable sensation that the cold pack generates against the anatomical area to which the cold pack is applied. For example, cold packs that have been subjected to extremely low and freezing temperatures may be so cold that their touch against exposed skin of the user is considered painful or unbearable to the user. Other users find the textile feel of the synthetic bag of the cold pack against their skin to be unpleasant. As a consequence, the cold packs are sometimes forsaken entirely or used ineffectively.

### SUMMARY OF THE INVENTION

According to a first aspect of the invention, a flexible band conformable into an annulus is provided. The flexible band comprises an outer layer made of a first thermally insulating material, and an inner layer made of a second thermally insulating material. The inner and outer layers are connected to one another to establish a pocket associated with a mouth sized to permit insertion and removal of a thermal pack through the mouth for accommodation in the pocket. The inner layer possesses a plurality of vent ports for permitting thermal transfer through the inner layer.

A second aspect of the invention provides a thermal cooling apparatus which comprises a thermal pack and a flexible band conformable into an annulus. The flexible band comprises an outer layer made of a first thermally insulating material and an inner layer made of a second thermally insulating material. The inner and outer layers are connected to one another to establish a pocket sized to accommodate the thermal pack. The pocket is associated with a mouth sized to permit insertion and removal of the thermal pack through the mouth for receipt in the pocket. The inner layer possesses a plurality of vent ports for permitting thermal transfer from the thermal pack through the inner layer.

A third aspect of the invention provides a method of treating an individual. According to this method, a thermal cooling apparatus is provided which comprises a thermal pack and a flexible band conformable into an annulus. The flexible band has inner and outer layers, with the inner layer having a plurality of vent holes. The thermal pack is inserted through a mouth of the thermal cooling apparatus into a cavity between the inner and outer layers of the flexible band. The inner layer is placed into contact with an anatomical area of a user, and the vent holes in the inner layer permit the thermal transfer from the thermal pack to the anatomical area. Preferably, the flexible band is wrapped around the anatomical area of the user and secured thereto, such as with a fastener, in the shape of an annulus.

According to a fourth aspect of the invention, a flexible band conformable into an annulus is provided. The flexible band comprises an outer layer made of a first thermally insulating, water-resistant material, and an inner layer made of a second thermally insulating, water-resistant material. The inner and outer layers are connected to one another to establish a pocket associated with a mouth sized to permit insertion and removal of a thermal pack through the mouth for receipt in the pocket. The flexible band further includes a fastener for attaching opposite end portions of the flexible band to one another to retain the flexible band conformed into the annulus. The fastener is adjustable to provide variable diameters to the annulus.

A fifth aspect of the invention provides a thermal cooling apparatus comprising a thermal pack, and a flexible band conformable into an annulus. The flexible band comprises an outer layer made of a first thermally insulating, water-resistant material, an inner layer made of a second thermally insulating, water-resistant material, and an adjustable fastener. The inner and outer layers are connected to one another to establish a pocket associated with a mouth sized to permit insertion and removal of the thermal pack through the mouth for accommodation in the pocket. The fastener attaches opposite end portions of the flexible band to one another to retain the flexible band conformed into the annulus. The fastener is adjustable to provide variable diameters to the annulus.

A sixth aspect of the invention provides a method of treating an anatomical area of an individual. According to this method, a thermal cooling apparatus is provided which comprises a thermal pack and a flexible band conformable into an annulus. The flexible band comprises an outer layer made of a first thermally insulating, water-resistant material, an inner layer made of a second thermally insulating, water-resistant material, and an adjustable fastener. The thermal pack is inserted through a mouth of the thermal cooling apparatus into a cavity between inner and outer layers of the flexible band. The inner layer is placed into contact with an anatomical area of a user, and the flexible band is wrapped around the anatomical area of the user and secured thereto via the fastener in the shape of an annulus.

### BRIEF DESCRIPTION OF THE DRAWINGS

The accompanying drawings are incorporated in and constitute a part of the specification. The drawings, together with the general description given above and the detailed description of the preferred embodiments and methods given below, serve to explain the principles of the invention. In such drawings:

FIG. 1 is a disassembled, perspective view of a thermal cooling apparatus according to an embodiment of the invention;

FIG. 2 is a cross-sectional view of the thermal cooling apparatus of FIG. 1 in an assembled state, taken along sectional line 2-2; and

FIG. 3 is a perspective view of the thermal cooling apparatus of FIG. 1 worn by an individual for cooling purposes.

### DETAILED DESCRIPTION OF PREFERRED EMBODIMENTS AND PREFERRED METHODS OF THE INVENTION

Reference will now be made in detail to the presently preferred embodiments and methods of the invention as illustrated in the accompanying drawings, in which like reference characters designate like or corresponding parts throughout the drawings. It should be noted, however, that the invention in its broader aspects is not limited to the specific details, representative devices and methods, and illustrative examples shown and described in this section in connection with the preferred embodiments and methods. The invention according to its various aspects is particularly pointed out and distinctly claimed in the attached claims read in view of this specification, and appropriate equivalents.

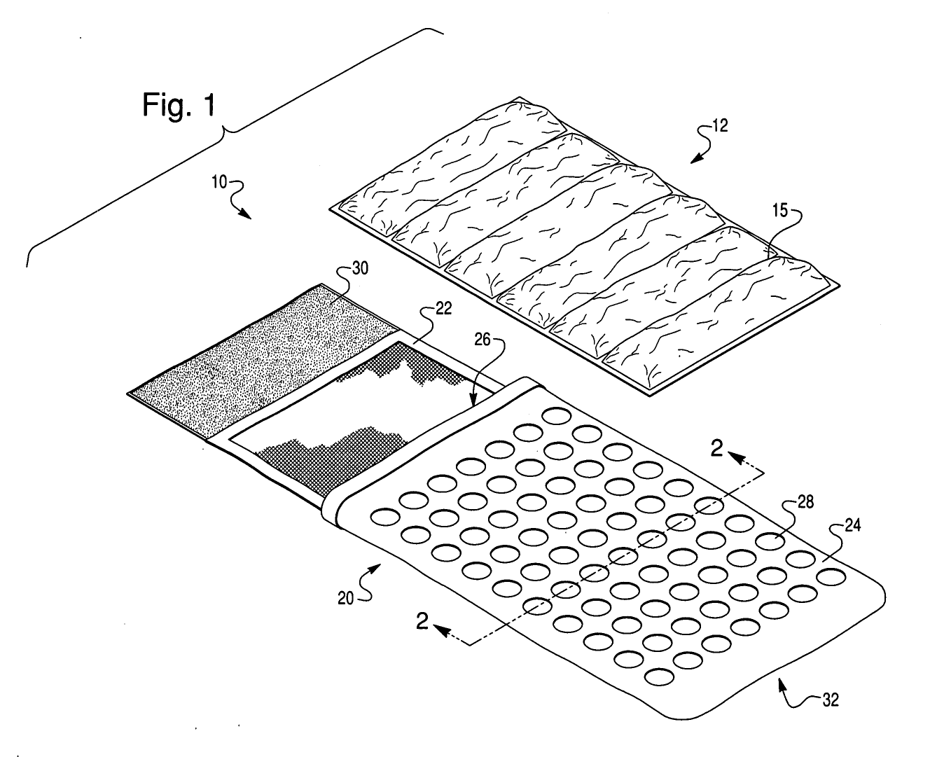

FIGS. 1-3 illustrate an embodiment of a thermal cooling apparatus, generally designated by reference numeral 10. Thermal cooling apparatus 10 includes a thermal pack 12 and a flexible band 20.

Thermal pack 12 may comprise a cold pack, a hot/cold pack, an instant cold compress, an instant hot compress, or other therapeutic products and materials that are capable of delivering reduced and/or elevated temperatures to an anatomical area of a user. Preferably, thermal pack 12 is a reusable product containing an insulating material which may be cooled or frozen in a refrigerator or freezer, then gradually warms when applied to an anatomical area of a user until the material returns to ambient temperature. Examples of insulating materials are gels, colloidal dispersions, and liquids such as, for example, water. Alternatively, thermal pack 12 may employ chemical components that, when activated, dissolve or otherwise react, for example, endothermically in the case of cold packs.

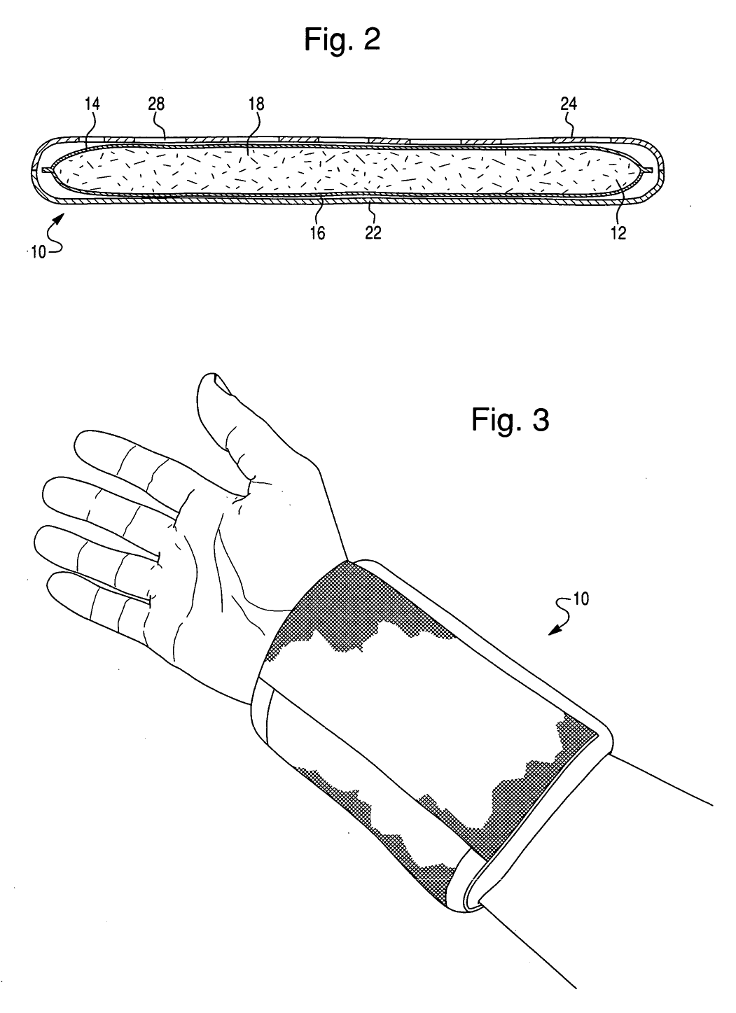

Thermal pack 12 has internal seams or welds 15 connecting opposite walls 14, 16 (FIG. 2) of thermal pack 12 to partition the cavity between walls 14, 16 into a plurality of compartments 18 isolated from one another. The subdivision of the cavity into compartments 18 improves the uniform distribution of the insulating gel or other contents within thermal pack 12, e.g., so that the contents of the cavity does not collect at an edges or corner of thermal pack 12. The isolated compartments 18 also assist in allowing thermal pack 12 to form an annulus in use, because seams 15 facilitate bending of thermal pack 12. It should be understood, however, that designs other than that shown in FIG. 1 may be implemented. For example, the cavity of thermal pack 12 may be divided into fewer or more compartments 18 than is shown in FIG. 1. Compartments 18 may be subdivided laterally, e.g., at the midpoint of compartments 18. Opposite walls 14, 16 may be flexible or rigid.

Flexible band 20 includes an outer layer (or main body) 22 having an interior surface and an exterior surface. Flexible band 20 further includes an inner layer (or pouch) 24 with an outward-facing surface in facing relationship with the interior surface of outer layer 22. Outer and inner layers 22, 24 may be co-extensive in length or one of the layers may be longer and/or wider than the other layer. For example, as best shown in FIG. 1, in the illustrated embodiment one of the ends of outer layer 22 extends beyond the corresponding end of inner layer 24 to provide a flange region.

The peripheral edges of outer layer 22 and inner layer 24 are preferably sewn to one another. Mechanical fasteners, adhesive bonds, or other techniques may be used in lieu of or in combination with the sewing. It is also possible for outer and inner layers 22, 24 to be integrally connected, e.g., formed as a unitary piece. Preferably, however, at least one set of edges of outer and inner layers 22, 24 is not connected. As best shown in FIG. 1, in the illustrated embodiment one of the ends of inner layer 24 is unattached to the interior surface of outer layer 22, i.e., proximal to the flange portion, to provide a mouth (or opening) 26 between the corresponding ends of outer and inner layers 22, 24. Mouth 26 is sized and configured to permit thermal pack 12 to be inserted through mouth 26, preferably without folding or bending thermal pack 12, which may be in a frozen solid state when inserted. Similarly, an open pocket (unnumbered) established between interior surface of outer layer 22 and an outward-facing surface of inner layer 24 is sized and configured to accommodate thermal pack 12, preferably without requiring that thermal pack 12 be folded or bent while retained in the pocket.

It should be understood that mouth 26 may be positioned at other locations, and may be formed in either outer layer 22 or inner layer 24, instead of at an interface of the layers 22, 24. Although not shown, a flap or non-permanent fastener, such as hook-and-loop fabric material (e.g., Velcro®), button, tie, etc., may be situated at mouth 26 to promote retention of thermal pack 12 within the pocket, yet to allow selective access to pocket for the purpose of inserting and removing thermal pack 12 at will, and without requiring the assistance of any tools or ripping of fabric or threads to access the pocket. Consequently, thermal pack 12 may be separated from flexible band 20, for example, for the purpose of returning a used thermal pack 12 into a refrigerator, freezer, microwave, or other temperature-regulating apparatus for later reuse in the pocket.

Outer layer 22 and inner layer 24 are preferably made of a material that is both thermally insulating and water resistant. A particularly preferred material is fabric-coated synthetic rubbers, such as rubbers based on polychloroprene, e.g., Neoprene® made by DuPont. Neoprene® is well known for its use in wetsuits and for electrical insulation. It is within the scope of the invention to select alternative materials for outer and inner layers 22, 24. Also, outer and inner layers 22, 24 may be made from the same or different materials. The provision of a thermally insulating material for outer layer 22 reduces the heat transfer of thermal pack 12 with the surrounding environment, and as a result improves and prolongs the effectiveness of thermal pack 12.

The selection of a thermally insulating material as inner layer 24 prevents inner layer 24 from being cooled to the same temperature as thermal pack 12. The inner layer 24, which is in direct contact with the user's skin, thereby remains at a temperature that the user will find comfortable and unobjectionable. The selection of a material that is also water resistant, particularly for inner layer 24, is also preferable to prevent inner layer 24 from becoming satiated with moisture, for example from condensation or due to sweat from the user. Maintaining an unsaturated, at least partially dry inner layer 24 avoids excessive thermal transfer directly through the material of inner layer 24, and circumvents a tactile unacceptable sensation comparable to that of wearing wet or sweat-soaked apparel.

Inner layer 24 includes a plurality of vent ports 28 for permitting thermal transfer from thermal pack 12 through inner layer 24 to the anatomical area of the user in contact with inner layer 24. The provision of vent ports 28 is particularly important for allowing thermal transfer where inner layer 24 is made of a thermally insulating material. It is preferred that vent ports 28 have a minimal cross-dimension in a range of from about ⅜ inch to about ½ inch, although sizes outside these ranges may be employed. For example, the minimum cross-dimension of a rectangle would be the distance between the opposite longer sides, i.e., the length of the shorter sides. Preferably, the area of inner layer 24 defining vent ports 28 is about 0.5 to about 3 mm in thickness, more preferably about 0.5 mm in thickness.

The illustrated vent ports 28 possess a circular shape with a constant minimum cross-dimension, which in the case of a circle is the diameter. It should be understood that vent ports 28 may possess other curvilinear, rounded shapes, such as that of an oval or eclipse, wherein the minimal cross-dimension is the minor axis. Alternatively vent ports 28 may possess polygonal shapes, such as that of a triangle, rectangle, square, pentagon, etc. Vent ports 28 may also possess elongated shapes, random shapes, and other shapes. Vent ports 28 may be the same or different from one another in size and shape. In the illustrated embodiment, vent ports 28 are shown in a matrix of defined columns and rows. It should be understood that other patterns as well as random arrangements of vent ports 28 may be employed.

As best shown in FIG. 3, flexible band 20 preferably is conformable into an annulus or ring-like shape. This high conformability allows flexible band 20 to be wrapped around various anatomical parts of the user, such as the wrist, forearm, upper arm, calf, ankle, and forehead.

A major advantage of the illustrated embodiment of the invention is that flexible band 20 may be adapted to fit around and reliably secured to various size body parts. This feature makes thermal cooling apparatus 10 suitable for use for both children and adults of various sizes and shapes, and for multiple body parts of different sizes. This advantage is made possible as the result of both the high conformability of flexible band 20 designed to provide variable control over the diameter of the annulus, and an adjustable fastener which allows thermal cooling apparatus 10 to be securely retained in its various annulus diameters, preferably while apparatus 10 is wrapped about an anatomical area of an individual. As illustrated, the adjustable fastener comprises a hook-and-loop material commonly referred to and sold under the trademark Velcro®. A strip with an interior surface of hook material 30 is attached at a first end portion of flexible band 20, and complementary loop material 32 is stitched or otherwise provided on the exterior surface of flexible band 20 at an opposite second end portion of flexible band 20. At least one, and preferably both of hook material 30 and loop material 32 are extensive in length to allow a wide degree in variation in the size of the annulus created and secured upon bringing the opposite end portions into interfacing relationship with one another.

It should be understood that loop material 32 may be placed on the interior surface of the strip, and that hook material 30 may be placed on the exterior surface of flexible band 20. Similarly, materials 30, 32 may be placed on the exterior surface of the strip and the interior surface of flexible band 20. It should be further understood that alternative or supplementary fasteners may be used, such as mechanical fasteners, for example, buckles, clips, buttons, tie strings, etc.

In use, a cold thermal pack 12 is removed from the refrigerator or freezer after being cooled to a desired temperature, or a hot thermal pack 12 is removed from a microwave, etc. after being heated to a desired temperature. Thermal pack 12 is inserted through mouth 26 of flexible band 20 into the pocket established between outer layer 22 and inner layer 24. Thermal cooling apparatus 10 is then applied to the anatomical area of the user by the user himself, or by another person, such as a trainer or physician. Inner layer 24 faces inward to contact against the skin of the user. As inner layer 24 contacts the skin of the user, the thermal pack 12 preferably is separated from contact from the skin by the thickness of inner layer 24. Simultaneously, vent ports 28 allow for the transfer of heat or cold from thermal pack 12 to the anatomical area.

The opposite ends of flexible band 20 are wrapped around the body part and adjusted to provide a snug, but preferably not restrictive fit. Hook material 30 on the strip engages against and secures complementary loop material 32 to retain thermal cooling apparatus 10 in place, freeing up the hands of the wearer to perform other tasks during treatment. Once thermal pack 12 returns to or near ambient temperature, often within a period of 1 hour or more, depending upon various factors, thermal pack 12 is removed from the pocket through mouth 26, and is returned to its source, e.g., refrigerator, freezer. Multiple thermal packs 12 may be used with a single flexible band 20. For example, multiple thermal packs 12 may be stored in a freezer, and used consecutively. That is, a cooled thermal pack may be taken from, for example, the freezer to replace a used thermal pack that has returned to ambient temperature and requires re-cooling. The used thermal pack may be returned to the freezer for later reuse. In this manner, thermal packs may be cycled with one another, and protracted delays in waiting for a used thermal pack to cool in the freezer are avoided.

Thermal cooling apparatus 10 is primarily intended for use in treatment of human beings, although apparatus 10 may also be used for veterinary purposes as well. It is also possible to use thermal cooling apparatus 10 for non-therapeutic applications, such as to cool or warm a beverage in a can, bottle, or glass.

The foregoing detailed description of the preferred embodiments of the invention has been provided for the purposes of illustration and description, and is not intended to be exhaustive or to limit the invention to the precise embodiments disclosed. The embodiments were chosen and described in order to best explain the principles of the invention and its practical application, thereby enabling others skilled in the art to understand the invention for various embodiments and with various modifications as are suited to the particular use contemplated. It is intended that the scope of the invention cover various modifications and equivalents included within the spirit and scope of the appended claims.

What is claimed is:

1. A flexible band conformable into an annulus, comprising:
an outer layer made of a first thermally insulating material; and
an inner layer made of a second thermally insulating material, said inner and outer layers connected to one another to establish a pocket including a mouth sized to permit insertion and removal of a thermal pack through said mouth for receipt in said pocket, said inner layer possessing a plurality of vent ports for permitting thermal transfer through said inner layer.

2. A thermal cooling apparatus, comprising:
a thermal pack; and
said flexible band of claim 1, wherein said pocket of the flexible band is sized to accommodate said thermal pack, said mouth of said flexible band is sized to permit insertion and removal of said thermal pack through the mouth, and said plurality of vent ports permit thermal transfer from said thermal pack through said inner layer.

3. The thermal cooling apparatus of claim 2, wherein at least one of said first and second materials comprises a fabric-coated neoprene.

4. The thermal cooling apparatus of claim 2, wherein said first and second materials are water resistant.

5. The thermal cooling apparatus of claim 2, further comprising a fastener for attaching opposite end portions of said flexible band to one another to retain said flexible band conformed into the annulus.

6. The thermal cooling apparatus of claim 5, wherein said fastener is adjustable to provide and secure the annulus at variable diameters.

7. The thermal cooling apparatus of claim 6, wherein said fastener comprises a hook-and loop fabric fastener.

8. The thermal cooling apparatus of claim 2, wherein said vent ports have a circular shape.

9. The thermal cooling apparatus of claim 2, wherein said vent ports have a minimum cross-dimension of 5 to 20 mm.

10. A method of treating an anatomical area of an individual, comprising:
providing a thermal cooling apparatus comprising a thermal pack and a flexible band conformable into an annulus, the flexible band having inner and outer layers with the inner layer having a plurality of vent holes;
inserting the thermal pack is inserted through a mouth of the thermal cooling apparatus into a cavity between the inner and outer layers of the flexible band;
placing the inner layer into contact with an anatomical area of an individual; and
permitting thermal transfer from the thermal pack through the vent holes to the anatomical area.

11. The method of claim 10, further comprising wrapping the flexible band around the anatomical area of the user, and securing the flexible band thereto in the shape of an annulus with the inner layer facing inward.

12. The method of claim 11, wherein the thermal cooling apparatus further comprises a fastener for attaching opposite end portions of the flexible band to one another to retain the flexible band conformed into the annulus.

13. The method of claim 12, wherein said fastener is adjustable to provide and secure the annulus at variable diameters.

14. The method of claim 10, wherein at least one of said inner and outer layer is made of a fabric-coated neoprene.

15. A flexible band conformable into an annulus, comprising:
an outer layer made of a first thermally insulating, water-resistant material;
an inner layer made of a second thermally insulating, water-resistant material, said inner and outer layers connected to one another to establish a pocket including a mouth sized to permit insertion and removal of a thermal pack through said mouth for receipt in said pocket; and
a fastener for attaching opposite end portions of said flexible band to one another to retain said flexible band conformed into the annulus, said fastener being adjustable to retain the annulus at variable diameters.

16. The flexible band of claim 15, wherein said first and second materials comprise a fabric-coated neoprene.

17. A thermal cooling apparatus, comprising:
a thermal pack; and
the flexible band of claim 15.

18. The thermal cooling apparatus of claim 17, wherein at least one of said first and second materials comprises a fabric-coated neoprene.

19. A method of treating an anatomical area of an individual, comprising:
providing the thermal cooling apparatus of claim 17;
inserting the thermal pack through the mouth of the thermal cooling apparatus into the cavity between the inner and outer layers of the flexible band;
contacting the inner layer of the thermal cooling apparatus with an anatomical area of an individual;
wrapping the flexible band around the anatomical area of the individual; and
securing the flexible band in the shape of an annulus around the anatomical area with the variable fastener.

20. The method of claim 19, wherein at least one of said inner and outer layer is made of a fabric-coated neoprene.
